(19) United States
(12) Patent Application Publication
(10) Pub. No.: US 2006/0241205 A1
(43) Pub. Date: Oct. 26, 2006

(54) Filler materials for dental composites

(76) Inventor: Weitao Jia, Wallingford, CT (US)

Correspondence Address: Ann M. Knab;Pentron Corporation, 53 North Plains Industrial Road, Wallingford, CT, 06492, US

(21) Appl. No.: 11/403,971

(22) Filed: Apr. 13, 2006

Related U.S. Application Data
(60) Provisional application No. 60/673,151, filed on Apr. 20, 2005.

(51) Int. Cl. A61K 6/083

(52) U.S. Cl. 523/115

(57) ABSTRACT

A dental composite comprising a reinforcing material in the form of glass flakes. The resulting filler material can be used in dental composites and dental restorations including but not limited to fillings, orthodontic retainers, bridges, space maintainers, tooth replacement appliances, dentures, crowns, posts, jackets, inlays, onlays, facings, veneers, facets, implants, abutments, cements, bonding agents and splints, to provide optimal handling properties, good wear resistance and high strength.

### TECHNICAL FIELD

The present invention relates generally to filler materials and methods of manufacture thereof and more specifically to filler materials for use in dental composite materials.

### BACKGROUND OF THE INVENTION

Compositions useful for repairing damaged teeth in situ are known in the art as direct filling materials, and include alloys and resin composites. Amalgam dental fillings are being increasingly replaced with dental composites that more closely match the color and appearance of the natural tooth. These composites generally consist of an organic resin that contains a microparticle filler. Most systems incorporate a light- or UV-curable polymeric resin, such as a diglycidylmethacrylate of bisphenol A (BIS-GMA), triethyleneglycol dimethacrylate (TEGDMA) or a urethane dimethacrylate (UDMA). The filler particles are typically barium silicate glass, quartz, or zirconium silicate, combined with small colloidal silica particles. Some composite formulations have also included glass fibers to further improve the properties of the resin materials. The glass fiber materials, however, tend to have smooth cylindrical surfaces which often create some difficulty for adherence of the resin matrix to the fiber surfaces, even when the fiber surfaces are treated with surface coupling agents.

Although fillers have added to the strength of dental composites, there is always need for improvement of fracture toughness, impact strength and dimensional stability. It would be advantageous to provide a composite with improved strength and fracture resistance while maintaining dimensional stability.

### SUMMARY OF THE INVENTION

These and other objects and advantages are accomplished by the composition and method of manufacture of the present invention directed to a dental composite comprising a reinforcing material in the form of glass flakes and a polymeric matrix material.

The resulting filler material can be used in dental composites and dental restorations including but not limited to fillings, orthodontic retainers, bridges, space maintainers, tooth replacement appliances, dentures, crowns, posts, jackets, inlays, onlays, facings, veneers, facets, implants, abutments, cements, bonding agents and splints, to provide optimal handling properties, good wear resistance and high strength.

### DETAILED DESCRIPTION OF THE INVENTION

The present invention is directed to a filler material that can be used to form dental composites and restorations in accordance with known procedures. The filler material of the present invention is a reinforcing agent in the form of a glass flake. Dental composite materials having good physical properties are realized using the filler material of the present invention. Dental composites using the filler material of the present invention exhibit flexural strengths equal to or greater than about 120 MPa, a flexural modulus equal to or greater than about 10 GPa and a Vickers hardness equal to or greater than about 500 MPa.

A number of compositions of the glass flakes are suitable for use in the practice of the present invention, including but not being limited to known glass flake materials listed in Table 1 below. Examples of two preferred commercially available glass flakes include Microglas® Glass Flakes available from NGF Canada Limited based in Guelph, Ontario.

TABLE 1
Glass Composition of Flakes

| Type of | | | | | | Na₂O + | |
|---|---|---|---|---|---|---|---|
| glass | SiO₂ | Al₂O₃ | CaO | MgO | B₂O₃ | K₂O | ZnO |
| C-glass | 65˜72 | 1˜7 | 4˜11 | 0˜5 | 0˜8 | 9˜13 | 0˜6 |
| (%) | | | | | | | |
| E-glass | 52˜56 | 12˜16 | 16˜25 | 0˜6 | 5˜13 | 0˜0.8 | |
| (%) | | | | | | | |

The glass flakes used in the present invention can be any standard size and preferably are between about 5 to about 1000 microns in length/width and more preferably are between about 15 to about 600 microns in length/width. The average thickness of the glass flakes can be any standard thickness. It is preferable that the thickness of the flakes is from about 1 to about 10 microns and more preferably from about 3 to about 7 microns in thickness. The specific gravity of the glass flakes is approximately 2.5.

The glass flakes may further be etched and/or treated with a coupling agent such as a silane compound which is known in the art to provide coupling between materials, as taught in commonly assigned U.S. Pat. Nos. 5,444,104, 4,547,531 and 4,544,359 all to Waknine, which are hereby incorporated by reference.

The polymeric matrix portion of the dental composite is selected from those known in the art of dental materials, including those listed in commonly assigned U.S. Pat. Nos. 6,013,694, 6,270,562, and 6,787,629 all to Jia and all of which are incorporated by reference herein. The polymeric matrix materials include, but are not limited to, expandable monomers, liquid crystal monomers, ring-opening monomers of epoxide resins, polyamides, acrylates, polyesters, polyolefins, polymides, polyarylates, polyurethanes, vinyl esters or epoxy-based materials. Other polymeric matrices include styrenes, styrene acrylonitriles, ABS polymers, polysulfones, polyacetals, polycarbonates, polyphenylene sulfides, and the like. These polymeric matrices are derived from curing polymeric matrix precursor compositions. Such precursor compositions are well-known in the art, and may be formulated as one-part, two-part, or other compositions, depending on the components.

Preferred materials include those based on acrylic and methacrylic monomers, for example those disclosed in U.S. Pat. Nos. 3,066,112, 3,179,623, and 3,194,784 to Bowen; U.S. Pat. Nos. 3,751,399 and 3,926,906 to Lee et al.; and commonly assigned U.S. Pat. No. 5,276,068 to Waknine and U.S. Pat. No. 5,969,000, all of which are herein incorporated by reference in their entirety. Especially preferred methacrylate monomers include the condensation product of bisphenol A and glycidyl methacrylate, 2,2′-bis[4-(3-methacryloxy-2-hydroxy propoxy)-phenyl]propane (hereinafter abbreviated BIS-GMA), the condensation product of ethoxylated bisphenol A and glycidyl methacrylate, (hereinafter EBPA-DMA), the condensation product of 2 parts hydroxymethylmethacrylate and 1 part triethylene glycol bis(chloroformate) (hereinafter PCDMA) and polycarbonate modified-BisGMA (PCBisGMA). Urethane dimethacrylate (UDMA), polyurethane dimethacrylates (hereinafter abbreviated to PUDMA), and diol dimethacrylates such as butanedimethacrylate, dodecanedimethacrylate, or 1,6-hexanedioldimethacrylate (HDDMA) are also commonly-used principal polymers suitable for use in the present invention.

The polymeric matrix precursor composition may further comprise a co-polymerizable diluent monomer. Such monomers are generally used to adjust the viscosity of the polymerizable composition, which affects wettability of the composition. Suitable diluent monomers include, without limitation, hydroxyalkyl methacrylates, such as 2-hydroxyethyl methacrylate, 1,6-hexanediol dimethacrylate, and 2-hydroxypropyl methacrylate; glyceryl dimethacrylate; ethyleneglycol methacrylates, including ethyleneglycol methacrylate, diethyleneglycol dimethacrylate, triethyleneglycol dimethacrylate and tetraethyleneglycol dimethacrylate; or diisocyanates, such as 1,6-hexamethylene diisocyanate. Triethyleneglycol dimethacrylate (TEGDMA) is particularly preferred for use in the present invention.

The polymeric matrix precursor composition typically includes polymerization initiators, polymerization accelerators, ultra-violet light absorbers, anti-oxidants, fluorescent whitening agents, and other additives well known in the art. The polymer matrices may be visible light curing, self-curing, dual curing, and vacuum-, heat- and pressure-curable compositions as well as any combination thereof. Visible light curable compositions employ light-sensitive compounds such as benzil diketones, and in particular, dl-camphorquinone in amounts ranging from about 0.05 to 0.5 weight percent. UV absorbers are particularly desirable in the visible light curable compositions in order to avoid discoloration of the resin form any incident ultraviolet light. Suitable UV absorbers are the various benzophenones, particularly UV-9 and UV-5411 available from American Cyanamid Company, and benzotriazoles known in the art, particularly 2-(2′-hydroxy-5′-methylphenyl)benzotriazole, sold under the trademark TINUVIN P by Ciba-Geigy Corporation, Ardsley, N.Y., 2,4,6-Trimethylbenzoyldiphenylphosphine oxide (Lucirin TPO) in amounts ranging from about 0.05 to about 5.0 weight percent. An example of an antioxidant is butylated hydroxytoluene (BHT). UVITEX-OB is an example of a fluorescent agent available from Ciba Specialty Chemicals.

Polymerization accelerators suitable for use are the various organic tertiary amines well known in the art. In visible light curable compositions, the tertiary amines are generally acrylate derivatives such as dimethylaminoethyl methacrylate and, particularly, diethylaminoethyl methacrylate (“DEAEMA”), and the like, in an amount of about 0.05 to about 0.5 wt %. In the self-curing compositions, the tertiary amines are generally aromatic tertiary amines, preferably tertiary aromatic amines such as ethyl 4-(dimethylamino)benzoate (commonly known as “EDMAB”), 2-[4-(dimethylamino)phenyl]ethanol, N, N-dimethyl-p-toluidine (commonly abbreviated “DMPT”), bis(hydroxyethyl)-p-toluidine, triethanolamine, and the like. Such accelerators are generally present at about 0.5 to about 4.0 wt % in the polymeric component and particularly in amounts ranging from about 0.05 to 0.5 weight percent.

The heat and pressure curable compositions include, in addition to the monomeric components, a heat cure initiator such as benzoyl peroxide, 1,1 ′-azobis(cyclohexanecarbonitrile), or other suitable free radical initiators. Particularly suitable free radical initiators are lauroyl peroxide, tributyl hydroperoxide, AIBN and, more particularly benzoyl peroxide or 1,1 ′-azobis(cyclohexanecarbonitrile).

The total amount of filler is determined by the specific function of the filled materials, being in the range from about 5 to 95% by weight of the total composite composition and the polymeric matrix being in the range of from about 5 to about 95% of the total composite composition. Preferably, the composites of the present invention may also include additional inorganic and/or organic fillers or a mixtures thereof currently used in dental restorative materials. Additional fillers may include one or more of silica, silicate glass, quartz, barium silicate, strontium silicate, barium borosilicate, borosilicate, lithium silicate, amorphous silica, ammoniated or deammoniated calcium phosphate, alumina, zirconia, tin oxide and titania. Preferably, the additional filler is barium borosilicate in an amount between about 5% and about 85% by weight of the total composite composition. Examples of glass fillers include those barium borosilicate or other suitable glass fillers commercially available from Schott Electronic Packaging GmbH (Landshut, Germany) under the product codes of GM 27884, G018161, G018-159 or 8235. When used as a direct filling material, the improved high strength composite material is obtained by using about 5% to about 60% by weight resin, about 5 to about 80% by weight of filler comprising the glass flakes and from about 5 to about 85% by weight of other filler and more preferably, about 10% to about 50% by weight resin, about 10 to about 60 % by weight of filler comprising the glass flakes and from about 30 to about 80% by weight of other filler, for example barium borosilicate. A preferred composition comprises about 20% by weight of a resin mixture, about 40% by weight of ground, glass flakes filler and 40% by weight of barium borosilicate filler.

In an alternative embodiment, the glass flakes may be further heat treated as in commonly assigned U.S. Pat. Nos. 6,013,694 and 6,403,676 to Jia, which are hereby incorporated by reference. Heat treatment involves densifying and embrittling the glass flakes by heating the glass flakes at a temperature substantially below the softening point of the glass flakes. Glass flakes as ordinarily provided by the manufacturer tend to be less dense, and somewhat flexible, that is, capable of being bent without breaking. As used herein, the term “densify” means to cause the flakes to become more dense, that is, to shrink in volume with practically no fusing or melting together of the flakes at their points of contact. “Embrittled” as used herein means to cause the flakes to become more prone to breakage upon the application of force, with practically no fusing or melting together of the flakes at their points of contact. Further as used herein, the term “substantially below” refers to a temperature effective to densify and embrittle the glass flakes, but not to fuse or melt the glass flakes together at their points of contact. The densified, embrittled glass particles are then cooled and ground to a particle size, depending on the starting flake size, to a size suitable for use in dental restorations.

In the practice of this alternative embodiment, glass flakes are densified and embrittled by heating at a temperature substantially below the softening point of the glass for a time effective to densify the glass. Such temperature and time are interdependent, and are empirically determined, based on the composition (and thus the softening point) of the glass. Higher temperatures will generally result in shorter times. The temperature must be high enough to effect densification and embrittlement, but not so high as to cause fusion, while the time of heating must be such as to allow even heating, but again, no fusion of the flakes at the chosen temperature.

In a preferred method of this alternate embodiment, at atmospheric pressure, the temperature is at least about 75° C. below the softening point of the glass, and more preferably, at atmospheric pressure, the temperature is between about 100° C. and about 140° C. below the softening point of the glass. C Glass, for example, has a softening point of 689° C. In accordance with the present invention, the C Glass flakes are preferably heated at a temperature between about 549° C. to about 580° C. for about 0.5 to 4 hours, a time period effective to densify the glass flakes but not fuse or melt the glass flakes together. In a particularly preferred embodiment, the C Glass flakes are heated at a temperature of about 560° C. for about 2 hours.

After densification, the glass flakes are ground to a size suitable for use as a filler in a dental restoration, preferably below about 100 microns. As used herein, grinding refers to any known methods for size reduction. The filler may be further treated with silane or other similar treatment.

In yet another embodiment herein, the glass flakes may be surface modified by bonding particles to the surface of the glass flakes. The surface-modifying particles are bonded to the glass flakes to increase its surface area and to improve the bonding properties of the flakes to enable them to better bond to a resin matrix material in a dental composite. Dental composite materials having good physical properties are realized using the filler material of the present invention. U.S. Pat. No. 6,270,562 is directed to a process of surface modifying fibers and is hereby incorporated by reference.

The surface-modifying particles, in general, can include any suitable filler material such as those set forth in commonly assigned U.S. Pat. Nos. 5,444,104, 4,547,531 and 4,544,359 all to Waknine, which are incorporated by reference herein. The surface modifying particles can include filler material which is capable of being covalently bonded to the resin matrix itself or to a coupling agent such as .gamma.-methacryloxy propyltrimethoxysilane which is available from OSi Specialties, Inc., Friendly, WV under the name Silquest A-174, which is covalently bonded to both the filler and the resin. Suitable surface-modifying particles include, but are not limited to, silica, silicate glass, quartz, barium silicate, strontium silicate, borosilicate, barium borosilicate, strontium borosilicate, lithium silicate, amorphous silica, ammoniated or deammoniated calcium phosphate, alumina, zirconia, tin oxide, titania, aluminum nitride, silicon nitride, titanium nitride, aluminum carbide, silicon carbide and titanium carbide. The particle size of the surface-modifying particles is preferably but not limited to the range of about 0.001 to about 5.0 microns depending upon the type of filler used, and more preferably is in the range of about 0.01 to about 1.0 micron. Preferably, the surface-modifying particles are smaller than the flakes such that the surface-modifying particles attach along the surface of the flakes.

The following non-limiting examples illustrate the invention.

### EXAMPLES

A polymerizable resin mixture set forth in Table 2 was used as the resin component of the resin-filler composite mixture. Glass flake fillers of the invention were used either alone, or in combination with another filler. Three different variations of flakes were used: 1) untreated; 2) silane-treated; and 3) heated, ground and silane-treated. A control sample having no glass flakes present was compared to the compostions having glass flakes. Commercially available Simile® composite from Pentron Clinical Technologies, LLC, Wallingford, Conn. was used as the control sample. The glass flakes were used as is, or further treated as described above, prior to mixing with the polymerizable resin mixture. The glass flakes employed in the Examples were C-Glass Microglas® glass flakes. The average thickness of the flakes was approximately 5 microns (um). Depending on the product used, the average width of the flakes was 15 um, 160 um, or 600 um. Table 3 sets forth the examples using the various fillers.

TABLE 2

| Resin Composition | Wt. Percent |
|---|---|
| PCBisGMA | 21.1 |
| BisGMA | 30.6 |
| UDMA | 23.5 |
| HDDMA | 23.1 |
| BHT | 0.02 |
| UV-5411 | 0.82 |
| Camphorquione | 0.2 |
| EDMAB | 0.44 |
| Lucirin TPO | 0.2 |
| UVITEX-OB | 0.02 |

TABLE 3

| | | | | Glass | Commercially | |
|---|---|---|---|---|---|---|
| | | | | Flakes | available | |
| | | | | heated, | silane treated | Commercially |
| | Polymerizable | | Glass | ground | glass filler, | available |
| | resin mix, | Glass | Flakes | and | 0.7 um | silane treated |
| | visible light | Flakes | with silane | silane- | average | amorphous |
| | curable | untreated¹ | treatment² | treated³ | particle size⁷ | silica⁷ |
| | (weight | (weight | (weight | (weight | (weight | (weight |
| Example | percent) | percent) | percent) | percent) | percent) | percent) |
| Control | 25 | | | | 75 | |
| Sample - | | | | | | |
| Simile ® | | | | | | |
| Composite | | | | | | |
| (Free of | | | | | | |
| Glass | | | | | | |
| Flakes) | | | | | | |
| A | 25 | 75⁴ | | | | |
| B | 45.5 | 54.5⁵ | | | | |
| C | 50 | 50⁶ | | | | |
| D | 16.7 | 33.3⁴ | | | 50 | |
| E | 16.7 | | 33.3⁴ | | 50 | |
| F | 19.2 | 19.3⁵ | | | 61.5 | |
| G | 18.5 | | 18.5⁵ | | 63 | |
| H | 16.4 | | 32.8⁴ | | 49.2 | 1.6 |
| I | 18.2 | | 18.2⁵ | | 61.8 | 1.8 |
| J | 16.4 | | | 32.8⁴ | 49.2 | 1.6 |
| K | 18.2 | | | 18.2⁵ | 61.8 | 1.8 |
| L | 18.9 | | | 18.9⁶ | 60.4 | 1.8 |

¹Used as is with no further treatment.
²The glass flakes were silane-treated as taught in U.S. Pat. No. 4,544,359 to Waknine, which is hereby incorporated by reference.
³The glass flakes were heat-treated, ground and silane-treated as taught in U.S. Pat. No. 6,013,694 to Jia, which is hereby incorporated by reference.
⁴C-Glass Glass Flakes from NGF Canada Limited having product code RCF 015 with average flake dimensions of about 15 microns in width and 5 microns in thickness.
⁵C-Glass Glass Flakes from NGF Canada Limited having product code RCF 160 with average flake dimensions of about 160 microns in width and 5 microns in thickness.
⁶C-Glass Glass Flakes from NGF Canada Limited having product code RCF 600 with average flake dimensions of about 600 microns in width and 5 microns in thickness.
⁷Product: Schott 8235
(8) Product: Degussa R7200

The various compositions were blended with a spatula by hand in a beaker, in proportions as indicated in Table 3. Example groups B and C were not able to test because the pastes formed were too loose and crumbly. The flexural strength and flexural modulus were tested according to ISO 4049: Dentistry—Resin based filling materials. The specimen dimensions were 25×2×2 mm. Specimens were prepared with a metal mold with glass slides on both the top and bottom of the mold. The material was filled into the mold and light cured for 2 minutes with a Cure-Lite™ Plus light curing box, Pentron Clinical Technologies, LLC, Wallingford, Conn.

Table 4 below shows the three-point bending flexural strength and flexural modulus results for the various example compositions.

TABLE 4

| | Flexural Strength, MPa | Flexural Modulus, GPa |
|---|---|---|
| Example | (S.D.) | (S.D.) |
| Control | 122.8 (3.6) | 11.2 (0.5) |
| A | 18.8 (1.4) | 2.4 (0.2) |
| B | — | — |
| C | — | — |
| D | 100.2 (3.3) | 11.5 (1.8) |
| E | 135.6 (9.3) | 12.0 (1.9) |
| F | 88.9 (5.1) | 12.9 (0.7) |
| G | 122.1 (4.9) | 14.7 (0.3) |
| H | 149.2 (7.3) | 12.0 (0.7) |
| I | 120 (7.9) | 14.2 (0.7) |
| J | 147.9 (3.7) | 11.5 (0.8) |
| K | 154.6 (6.4) | 10.1 (0.4) |
| L | 138.2 (13.4) | 9.9 (0.6) |

As it can be seen from the above results, the best results of various compositions containing the glass flake fillers are the combination of the glass flakes with a conventional glass filler of smaller particle size. In the examples herein, the use of barium-boro-silicate glass fillers with average particle sizes of 0.7 um has shown the synergy effect of the combination. Furthermore, when the glass flakes are further surface silane treated, or further subjected to heating, grinding and silane treatment before the use in a dental composite composition, the property of the composites containing the glass flakes can further be enhanced.

As will be appreciated, the present invention provides a filler composition having high strength and good bonding properties particularly useful in the fabrication of dental restorations.

While various descriptions of the present invention are described above, it should be understood that the various features can be used singly or in any combination thereof. Therefore, this invention is not to be limited to only the specifically preferred embodiments depicted herein.

Further, it should be understood that variations and modifications within the spirit and scope of the invention may occur to those skilled in the art to which the invention pertains. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. A dental restoration comprising:
glass flakes having a length/width in the range of about 15 to about 1000 microns and a thickness in the range of about 1 to about 10 microns; and
a polymeric matrix precursor composition.

2. The dental restoration of claim 1 wherein the glass flakes have a composition comprising about 65 to about 72% SiO2, about 1 to about 7% Al2O3, about 4 to about 11%CaO, up to about 5% MgO, up to about 8% B2O3, up to about 6% ZnO, and about 9 to about 13% Na2O+K2O.

3. The dental restoration of claim 1 wherein the glass flakes have a composition comprising in percent by weight about 52 to about 56% SiO₂, about 12 to about 16% Al₂O₃, about 16 to about 25% CaO, up to about 6% MgO, about 5 to about 13% B₂O₂, up to about 0.8% Na₂O+K₂O.

4. The dental restoration of claim 1 selected from the group consisting of fillings, orthodontic retainers, bridges, space maintainers, tooth replacement appliances, dentures, crowns, posts, jackets, inlays, onlays, facings, veneers, facets, implants, abutments and splints.

5. The dental restoration of claim 1 comprising an additional filler material.

6. The dental restoration of claim 1 further comprising polymerization initiators, polymerization accelerators, ultra-violet light absorbers, anti-oxidants, fluorescent whitening agents and mixtures thereof.

7. The dental restoration of claim 1 wherein the glass flakes are surface treated.

8. The dental restoration of claim 7 wherein the glass flakes are treated with silane.

9. The dental restoration of claim 1 wherein the glass flakes are heated, ground and treated with silane.

10. The dental restoration of claim 1 wherein the glass flakes are present in an amount from about 5 to about 95% of the composition and the polymeric matrix is present in an amount from about 5 to about 95% of the composition.

11. The dental restoration of claim 1 wherein the polymeric matrix comprises expandable monomers, liquid crystal monomers, ring-opening monomers of epoxide resins, polyamides, acrylates, polyesters, polyolefins, polymides, polyarylates, polyurethanes, vinyl esters, epoxy-based materials, styrenes, styrene acrylonitriles, ABS polymers, polysulfones, polyacetals, polycarbonates, polyphenylene sulfides, or mixtures thereof.

12. A dental composite composition for forming a restoration comprising:
ground, densified, embrittled glass flakes, wherein the flakes are derived from glass flakes heated at a temperature between about 100° C. and about 140° C. below the softening point of the glass flakes for a period of time effective to densify and embrittle the glass flakes; and
a polymeric matrix precursor composition.

13. The composite of claim 12 wherein the glass flakes have a composition comprising about 65 to about 72% SiO2, about 1 to about 7% Al2O3, about 4 to about 1 1%CaO, up to about 5% MgO, up to about 8% B2O3, up to about 6% ZnO, and about 9 to about 13% Na2O+K2O.

14. The composite of claim 12 wherein the glass flakes have a composition comprising about 52 to about 56% SiO₂, about 12 to about 16% Al₂O₃, about 16 to about 25% CaO, up to about 6% MgO, about 5 to about 13% B₂O₂, up to about 0.8% Na₂O+K₂O.

15. The composite of claim 12 wherein temperature is in the range between about 549° C. and about 580° C. .

16. The composite of claim 12 wherein the temperature is about 560° C. .

17. The composite of claim 12 wherein the polymeric matrix precursor composition comprises expandable monomers, liquid crystal monomers, ring-opening monomers of epoxide resins, polyamides, acrylates, polyesters, polyolefins, polymides, polyarylates, polyurethanes, vinyl esters, epoxy-based materials, styrenes, styrene acrylonitriles, ABS polymers, polysulfones, polyacetals, polycarbonates, polyphenylene sulfides, or mixtures thereof.

18. The composite of claim 12 wherein the glass flakes are present in an amount from about 5 to about 95% of the composition and the polymeric matrix is present in an amount from about 5 to about 95% of the composition.

19. The composite of claim 12 wherein the ground, densified, embrittled glass flakes comprise from about 5% to about 80% by weight of the total composite composition.

20. The composite of claim 12 wherein the ground, densified, embrittled glass flakes comprise about 35% by weight of the total composite composition.

21. The composite of claim 12 wherein the dental composite comprises at least one additional filler material.

22. The composite of claim 21 wherein the at least one additional filler material is at least one of silica, silicate glass, quartz, barium silicate, strontium silicate, barium borosilicate, borosilicate, lithium silicate, amorphous silica, ammoniated or deammoniated calcium phosphate, alumina, zirconia, tin oxide or titania.

23. The composite of claim 21 wherein the at least one additional filler material is barium borosilicate, comprising between about 5% to about 85% by weight of the total composite composition.

24. A dental restoration comprising:
glass flakes; and
a polymeric matrix precursor composition;
wherein the glass flakes comprise surface-modifying particles bonded to the glass flakes.

25. The dental restoration of claim 24 wherein the glass flakes have a composition comprising about 65 to about 72% SiO2, about 1 to about 7% Al2O3, about 4 to about 11%CaO, up to about 5% MgO, up to about 8% B2O3, up to about 6% ZnO, and about 9 to about 13% Na2O+K2O.

26. The dental restoration of claim 24 wherein the glass flakes have a composition comprising about 52 to about 56% SiO₂, about 12 to about 16% Al₂O₃, about 16 to about 25% CaO, up to about 6% MgO, about 5 to about 13% B₂O₂, up to about 0.8% Na₂O+K₂O.

27. The dental restoration of claim 24 wherein the surface-modifying particles are fabricated of at least one of silica, silicate, ammoniated or deammoniated calcium phosphate, alumina, zirconia, tin oxide, titania, aluminum nitride, silicon nitride, titanium nitride, aluminum carbide, silicon carbide and titanium carbide.

28. The dental restoration of claim 24 further comprising at least one additional filler material.

29. The dental restoration of claim 27 wherein the silica comprises quartz or amorphous silica.

30. The dental restoration of claim 27 wherein the silicate comprises barium silicate, strontium silicate, borosilicate, lithium silicate, or silicate glass.

31. The dental restoration of claim 30 wherein the borosilicate comprises barium borosilicate.

32. The method of claim 24 wherein the surface-modifying particles are fabricated of a glass, fumed silica or both.

33. The method of claim 32 wherein the glass has a composition comprising about 50% SiO.sub.2, 15% Al.sub.2 O.sub.3, 1% BaO, 20% SrO, and 15% B.sub.2 O.sub.3.
